(19) United States
(12) Patent Application Publication
(10) Pub. No.: US 2002/0095130 A1
(43) Pub. Date: Jul. 18, 2002

(54) Adhesive garment closures

(76) Inventors: Christopher R. Seitter, Hampton Falls, NH (US); William H. Shepard, Amherst, NH (US)

Correspondence Address: DANIEL J. SEPANIK, FISH & RICHARDSON P.C., 225 FRANKLIN STREET, BOSTON, MA, 02110-2804, US

(21) Appl. No.: 09/760,433

(22) Filed: Jan. 12, 2001

Related U.S. Application Data
(60) Provisional application No. 60/253,615, filed on Nov. 28, 2000.

(51) Int. Cl. A61F 13/15; A61F 13/20; A41D 1/00; A41D 3/02; A41B 1/10; A41D 19/00; A63B 71/14; A42C 5/04; A41D 1/14; A41D 1/06

(52) U.S. Cl. 604/389; 604/369; 604/390; 604/391; 604/385.31; 24/306; 24/704.1; 2/78.4; 2/100; 2/128; 2/141.1; 2/161.4; 2/171.4; 2/219; 2/235

(57) ABSTRACT

A self-adhesive garment closure for a garment includes a pair of opposed cooperating fastener elements, each having an exposed layer of self-adhesive material exhibiting strong adhesion to itself while remaining relatively tack-free to other materials. At least one of the fastener elements has a layer of foam material beneath the self-adhesive material, such as for increased bending stiffness. In some cases, such as for pull-up diapers, the self-adhesive closure is non-releasable once secured. Some closures also have cooperating hook-and-loop elements.

### TECHNICAL FIELD

This invention relates generally to adhesive closures for disposable garments, such as diapers, and more particularly to such closures with self-adhering materials.

### BACKGROUND

Disposable diapers are generally formed of a flexible sheet material that is secured to the wearer by diaper closures provided at the waist region of the diaper. Diaper closures often include a pressure sensitive adhesive or a hook-and-loop fastener. U.S. Pat. No. 5,378,536 discloses a diaper closure that includes a repositionable low tack or tack free adhesive including a blend of an elastomeric block copolymer with a tackifying material.

Other garments, for example incontinence devices., surgical gowns, hats or booties, clean room garments, ankle bands and wrist bands, may also include similar types of closures.

### SUMMARY

In most of its various aspects, the invention features self-adhesive closures for garments, and garments including such closures. Generally, the self-adhesive closures include opposed cooperating fastener elements, each fastener element having a self-adhesive surface. The term “self-adhesive”, as used herein, refers to a surface that exhibits adhesion to itself (e.g., when the surface is folded over on itself) and to surfaces having similar self-adhesive properties (e.g., when the surface is pressed against a cooperating fastener element), while exhibiting relatively weaker or no appreciable adhesion to dissimilar surfaces and contaminants. Preferred self-adhesive closures exhibit little or no adhesion to dissimilar surfaces and contaminants, and are substantially tack free.

According to one aspect of the invention, a garment closure includes a pair of opposed cooperating fastener elements. Each fastener element has a flexible, sheet-form backing and a layer of self-adhesive material carried upon the backing and exposed for engagement with the cooperating fastener element. At least one of the fastener elements has a flexible foam layer sandwiched between its backing and its layer of self-adhesive material.

In some preferred embodiments, one of the fastener elements is in the form of a graspable tab having one end configured for permanent mounting to the garment and extending to an opposite, free end. The self-adhesive material of the tab may be spaced apart from the free end of the tab to form a graspable end region free of the self-adhesive material, for example, or has an edge region nearest the free end and loose from the backing.

Preferably, the self-adhesive material substantially covers the flexible foam layer.

For some applications, the foam layer is a closed cell foam. For some other applications, such as those requiring porosity for breathability, open cell foams may be employed. In many cases, it is preferred that both fastener elements include a corresponding flexible foam layer sandwiched between their backings and layers of self-adhesive material Suitable flexible sheet materials include flexible plastic films, e.g., polyethylene, polypropylene, polyvinyl chloride, NYLON, and other flexible sheet materials such as fabrics, non-woven materials and papers. The flexible material should generally have sufficient tear strength so that, when the flexible material is bonded to a selected foam layer, the fastener will resist tearing and permanent deformation during normal use.

Suitable foams are those that have sufficient flexibility and strength to be used in a given application without interfering with proper operation of the fastener. In some applications, the foam should resist tearing under fastener opening loads. In other applications, the foam tear resistance should be selected to cause the foam to tear before the self-adhesive layers of the mating fastener separate, such as for tamper evident or single use closures. Suitable foams will also provide the closure with resiliency, to allow the closure surfaces to engage around contaminants and to allow lateral distortion of the closure without stressing of the bond. The thickness of the foam layer will depend upon the application in which the fastener is used. Thinner foam layers (e.g., with a thickness of 0.020 to 0.050 inch) are more readily run through conventional diaper and fastener assembly equipment.

Thicker foam layers (e.g., with a thickness of 0.050 to 0.125 inch) may be required for closures that are to be subjected to sharp distortions. Suitable foam materials include polypropylene, polyethylene and polyurethane, among others.

The foam layer may be applied to the flexible sheet material in any desired manner, e.g., by lamination, adhesion, or co-extrusion. For many garment applications, flexibility is a highly desirable characteristic, and preferred closures have an assembled Gurley stiffness of between about 25 and 100 milligrams, as measured in accordance with ASTM D6125-97, “Gurley Bending Resistance of Paper and Paperboard”.

The self-adhesive layer is formed of a material that provides a desired degree of self-adhesion, combined with minimal adhesion to dissimilar surfaces and contaminants. Generally, the self-adhesive layer is an elastomer that has been modified to provide self-adhesive properties. Suitable self-adhesive elastomers are described, e.g., in U.S. Pat. Nos. 4,791,024, 4,956,228 and 4,985,299, the disclosures of which are incorporated herein by reference. For many releasable garment closure applications, particularly those configured to be loaded primarily in shear, as is the case for many diaper tab closures, the optimum peel strength required of the closure is only about 200 to 400 grams, as tested according to ASTM D5170-98, for easy opening by an adult. Peel strength is augmented, in some embodiments, by leaving the outer edge of the fastener element loose from the graspable end of the closure tab, such that initial separation forces are converted into shear loads at the self-adhesive interface as the graspable tab is peeled away from the front of the diaper, for example. Closure shear strength is preferably between about 3,000 and 5,000 grams, as tested according to ASTM D5169-98, for diaper closures. The thickness of the self-adhesive layer will depend upon the properties required of the fastener, and the elastomer selected.

Preferably, the self-adhesive elastomer is a thermoset material, particularly for applications which may be subjected to elevated temperatures during storage, or which require particularly good dimensional stability. For the self-adhesive material to be suitable for repeated engagement cycles, the cohesive strength of the material (as well as the adhesion of the material to its carrier material, such as the foam layer) must be less than the force required to separate the two engaged layers of self-adhesive material. Furthermore, for a “tack-free” characteristic as regards adhering to other materials, the self-adhesive material should provide little to no adhesion (preferably, no measurable adhesion) to materials other than a contacting layer of similar material. As discussed in the reference patents incorporated above, a suitable adhesive material is an elastomer such as natural rubber dissolved in a solvent such as heptane (with a proportion, for example, of 15% natural rubber and 55% heptane). Another suitable adhesive is a mixture of elastomeric materials, such as natural rubber and butadiene-styrene rubber. Either natural or synthetic adhesives may be used, as the application allows.

For some applications in which the natural characteristics of a thermoset material are not required, a self-adhering adhesive in the form of a thermoplastic block copolymer, such as that disclosed in U.S. Pat. No. 5,378,536 (hereby incorporated herein by reference in its entirety), may be employed to advantage.

The self-adhesive layer may be applied to the foam layer using any suitable coating method, e.g., roll-coating or spraying.

For releasable fastening applications, the layer of self-adhesive material is releasably engageable with the cooperating fastener element, to provide a multiple cycle fastening. For unreleasable fastenings, the layers of self-adhesive material preferably have an adhesive strength, when engaged, selected to be greater than a functionally related tear strength of one of the fastener elements, to cause the closure to irreparably tear before separating the self-adhesive material layers. The functionally related tear strength may be of the flexible foam layer, for example.

In some embodiments, the closure also has mating hook-and-loop fastener regions adjacent the layers of self-adhesive material and arranged for releasable engagement when the layers of self-adhesive material are engaged.

According to another aspect of the invention, a garment includes a body of flexible sheet material, and a self-adhesive closure constructed to secure the body in place around a wearer during use. The self-adhesive closure includes a pair of opposed cooperating fastener elements, each fastener element having a flexible backing and a layer of self-adhesive material carried upon the backing and positioned for cooperating engagement with the self-adhesive material of the other fastener element. At least one of the fastener elements also has a flexible foam layer sandwiched between its backing and its layer of self-adhesive material.

The garment may be of a form selected from the group consisting of diapers, incontinence devices, surgical gowns, hats, booties, clean room garments, ankle bands and wrist bands, for example. In many preferred constructions, including those illustrated herein, the garment is a diaper.

In some embodiments, the diaper also includes a primary fastener for securing the diaper about the infant, with the self-adhesive closure being a secondary fastener. One of the fastener elements of the self-adhesive closure is disposed above the primary fastener on an outer surface of a front panel of the diaper, and the other of the fastener elements disposed adjacent an upper edge of an inner surface of a rear panel of the diaper. Such an arrangement can help to avoid side bunching at the waistline, for example.

In some embodiments, the fastener elements are disposed along lateral edges of the diaper, extending substantially between leg openings and upper diaper edges. In such cases it is preferred that the layers of self-adhesive material have an adhesive strength, when engaged, selected to be greater than a functionally related tear strength of one of the fastener elements, to cause the closure to irreparably tear before separating the self-adhesive material layers. It is also preferred that one of the fastener elements extends to a lateral edge of an inner surface of a back panel of the diaper, so as to secure the lateral edge of the inner surface of the back panel of the diaper against grasping when secured to the other of the fastener elements.

For non-openable garment closures, it is preferred that the layers of self-adhesive material have an adhesive strength, when engaged, selected to be greater than a functionally related tear strength of one of the fastener elements, such as the flexible foam layer, to cause the closure to irreparably tear before separating the self-adhesive material layers.

For use as a diaper closure, among others, one of the fastener elements may be in the form of a graspable tab having one end configured for permanent mounting to the garment and extending to an opposite, free end securable to the other of the fastener elements.

According to another aspect of the invention, a diaper includes a body of flexible sheet material forming front and rear panels with upper edges, a primary closure for securing the body in place around a wearer during use, and a secondary closure disposed between the primary closure and the upper edges of the front and rear panels. The primary closure has a graspable tab having one end configured for permanent mounting to the rear panel of the body and having a free end securable to the front panel of the body. The secondary closure includes a pair of opposed cooperating fastener elements, each fastener element having an exposed layer of self-adhesive material positioned for cooperating engagement with the self-adhesive material of the other fastener element.

Preferably, at least one of the fastener elements of the secondary closure further includes a flexible foam layer beneath its layer of self-adhesive material.

For non-openable closures, it is preferred that the layers of self-adhesive material have an adhesive strength, when engaged, selected to be greater than a functionally related tear strength of an adjacent portion of the diaper, to cause the diaper to irreparably tear before separating the self-adhesive material layers of the closure.

According to yet another aspect of the invention, a diaper includes a body of flexible sheet material forming front and rear panels with upper edges, and an unreleasable adhesive closure constructed to secure the body in place around a wearer during use. The adhesive closure has a pair of opposed cooperating fastener elements, each fastener element including an exposed layer of adhesive material positioned for cooperating engagement with the adhesive material of the other fastener element. The layers of adhesive material exhibit an adhesive strength, when engaged, selected to be greater than a functionally-related tear strength of an adjacent portion of the diaper, to cause the diaper to irreparably tear before separating the adhesive material layers of the closure.

Preferably, at least one of the fastener elements has a flexible foam layer beneath its layer of adhesive material. The functionally related tear strength may be of the flexible foam layer, for example.

In some arrangements, one of the fastener elements extends to a lateral edge of an inner surface of the rear panel of the diaper.

Preferably, the adhesive material of each fastener element is a self-adhesive material.

The foam layer gives the closure a desirable soft, compliant feeling, improving user comfort and enhancing the user's perception of the garment as comfortable and easy to put on. The foam layer can also provide the closure with soft edges, to prevent the user from experiencing discomfort when the edges of the closure contact the user's skin. The foam layer provides resiliency to enable the closure to seal around contaminants, such as particulate matter. Moreover, the foam allows for some lateral distortion of the closure without stressing the bond layer. When the garment closure is flexed, the foam layer can undergo shear distortion through its thickness (i.e., the inner and outer surfaces of the foam layer can be displaced laterally relative to one another) and thus reduce the shear load applied to the adhesive bonds.

Advantageously, some preferred closures of the invention are refastenable, repositionable, relatively easy to open and close, and are relatively resistant to contamination, e.g., by dust, sand and other particulate matter.

Other features and advantages of the invention will be apparent from the description and drawings.

### DESCRIPTION OF DRAWINGS

FIG. 1 is a schematic perspective view of a diaper including a self-adhesive closure.

FIG. 1A is a schematic perspective view of the diaper of FIG. 1 being fastened about an infant.

FIG. 1B is a highly enlarged detail view of a fastener tab of the diaper of FIG. 1.

FIG. 2 illustrates a second fastener tab construction.

FIG. 3 is a schematic perspective view of a diaper with a combination self-adhesive and touch fastener closure arrangement.

FIG. 4 is a schematic perspective view of another diaper, equipped with a different self-adhesive closure arrangement.

### DETAILED DESCRIPTION

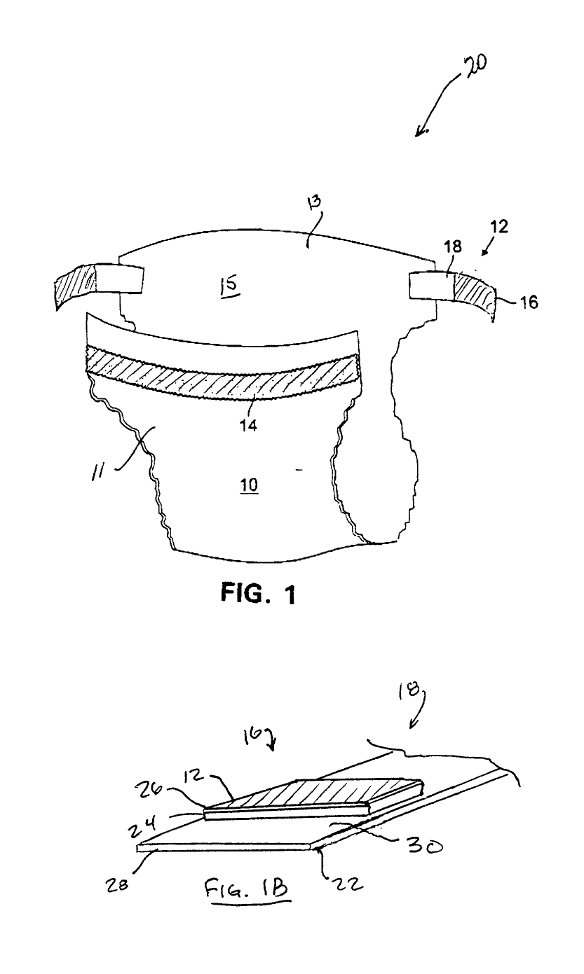

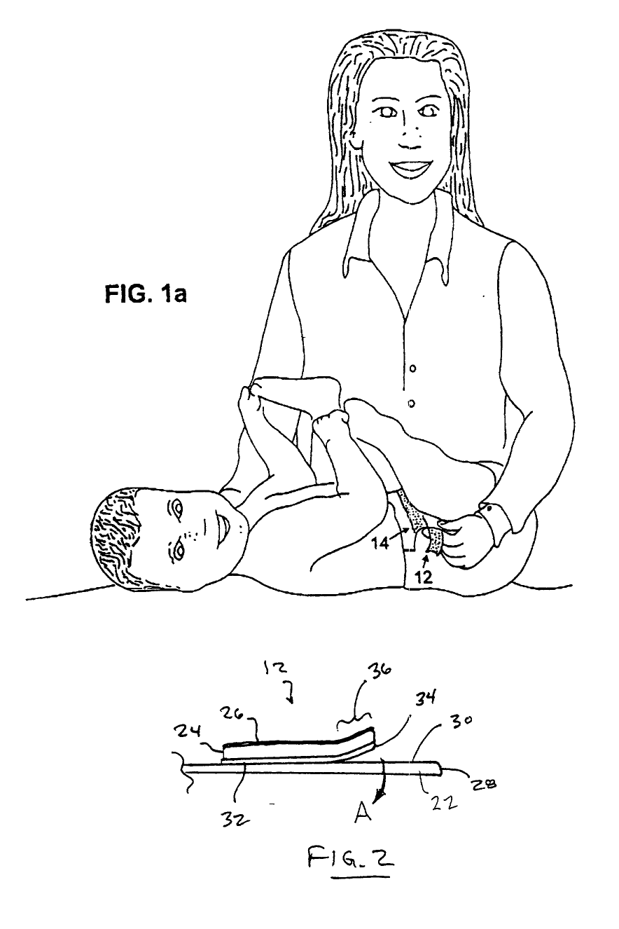

Referring to FIG. 1, a diaper 20 includes a pair of self-adhesive closures, each of which consists of cooperating self-adhesive fastener elements 12 and 14. Fastener elements 12 and 14 are each formed of a laminate 16, the structure of which will be described in detail below. Fastener element 12 is mounted on back portion 13 of the diaper on an extended tab 18 which is firmly secured to the inner surface 15 of the back portion, e.g., by heat sealing. Fastener element 14 is firmly secured to outer surface 10 of the front portion 11. Fastener element 14 may extend across the entire width of the front portion, as shown, or may consist of two patches that are positioned for cooperative engagement with fastener elements 12.

As shown in FIG. 1A, the diaper is fastened around the waist of an infant by overlapping the opposed fastener elements 12 and 14 to seal the self-adhesive closures and thereby fasten the corners of back portion 13 to the front portion 11. When secured, the adhesion between fastener elements 12 and 14 exhibits a peel strength of about 0.5 to 1.0 pound per lineal inch.

Laminate 16, shown in detail in FIG. 1B, includes a flexible sheet material 22, a 0.040 inch thick layer of foam 24 bonded to the flexible sheet material, and a layer of self-adhesive polymer 26 coated on the broad outer surface of foam 24. The layer of foam is spaced apart from the distal end 28 of the diaper tab a distance of about ¼ inch to provide a non-fastening, graspable region 30.

In another tab construction, shown in FIG. 2, self-adhesive fastener element 12 is laminated to a backing 32, which is then laminated to sheet material 22 save for an edge 34 facing tab distal end 28. This construction causes the corresponding edge region 36 of self-adhesive material overlying the unattached portion to flex away from substrate 22 upon application of a peeling separation load to the substrate (as, indicated by arrow A), to align the adhesive plane of engagement between the mated self-adhesive fastener elements with the separating load and thus place the adhesive bond in shear, rather than peel, over the unattached width of element 12.

The diaper 20a of FIG. 3 has tabs 18a that releasably engage the front panel 11a of the diaper by both adhesive and mechanical means. Panel 11a has a laminate 16 of foam and self-adhesive material, as described above, in the form of a strip 40 extending between two parallel strips of loop material 38. Each tab 18a has a similar region 42 of foam-backed self-adhesive material between two patches 44 of loop-engageable male fastener elements, such as molded hooks. When tabs 18a are stretched and placed against front panel 11 a as the diaper is secured on an infant, hook patches 44 releasably engage their corresponding strips of loop material 38 while the self-adhesive areas 40 and 42 adhere to one another. This combination-type closure can beneficially obtain performance advantages from each type of closure. For example, the hook-and-loop interface can provide enhanced peel resistance along the sides of the adhesive region, maintaining the integrity of the adhesive closure for resisting shear loads. To this end, the male fastener elements can be molded to extend across the width of the tab, for good cross-tab peel performance. An example of a preferred hook is shown in U.S. Pat. No. 6,131,251, the contents of which are incorporated herein by reference. Other types and shapes of male fastener elements, such as mushrooms, prongs, palm trees and other multi-lobal forms, may also be employed. The self-adhesive and touch fastener regions may also be disposed in other arrangements. For example, the regions may be separated along the length of the tabs, extending across the tabs rather than along their length. In one such example (not shown), a self-adhesive patch at the base of the tab provides good shear resistance when secured against a corresponding patch of self-adhesive material at the end of the front panel of the diaper, with a patch of hooks at the distal end of the tab engaging a loop region in the middle of the front panel. Such a configuration has the advantage of adhesive fastening without the need for handling the adhesive regions during diapering, as the hook patch would be covering the region of the tab normally grasped.

FIG. 3 also shows a secondary fastening means, including two small patch 46 of self-adhesive material at the upper corners of front panel 11a, positioned to engage two small corresponding strips 48 of similar material along the upper edge of the back panel of the diaper. With the diaper secured on an infant, this secondary fastening means keeps the upper edges of the diaper from sagging or curling down, enhancing leak protection and helping to prevent pinching at the hips. These mating patches may be constructed as foam laminates, as described above, to incorporate some bending resistance into the upper diaper edge corners and increase graspability. These secondary patches may also be constructed with relatively weak foam layers that noticeably tear when the diaper is unfastened, to provide evidence that a caregiver has dutifully checked the diaper, for example.

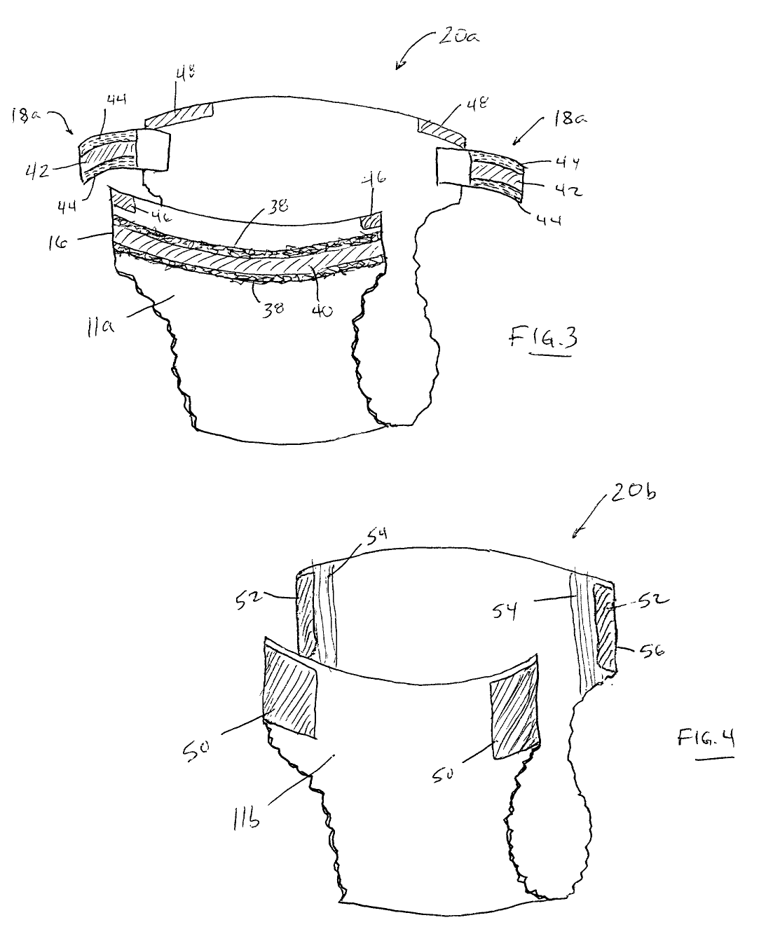

FIG. 4 illustrates a pull-up diaper 20b for older children, in which the fastening means is designed as a single-use, non-releasable closure. Vertical strips 52 of self-adhesive material along the side edges of the back panel of the diaper engage corresponding wide patches 50 of self-adhesive material at either end of front panel 11b to initially secure the diaper to the child and to provide a desired fit about the waist. Elasticity from other elements, such as elastic regions 54, maintains a residual shear load across the fasteners as the child moves about, and enables the diaper to be pulled down over the hips without releasing the fasteners. Adhesive patches 52 extend to the lateral edges 56 so as to not provide any free edge graspable by the child once secured. Both patches 50 and 52 have appreciable length along the lateral sides of the upper diaper panels, preferably from adjacent the elastic leg openings to adjacent the upper diaper edges, so as to provide bending resistance to keep the upper edges of the diaper from folding over and bunching during use. At least one of the matable self-adhesive surfaces may be covered with a release liner (not shown) until ready for use, to avoid unintended engagement.

Such closures are also useful in other disposable garments, such as hospital gowns, incontinence devices, hats or booties, clean room garments, ankle bands and wrist bands, and sanitary products.

Other embodiments are within the scope of the invention.

What is claimed is:

1. A closure for a garment, the closure including a pair of opposed cooperating fastener elements, each fastener element comprising a flexible, sheet-form backing and a layer of self-adhesive material carried upon the backing and exposed for engagement with the cooperating fastener element;
at least one of the fastener elements further comprising a flexible foam layer sandwiched between its backing and its layer of self-adhesive material.

2. The closure of claim 1 wherein the backing is a plastic film.

3. The closure of claim 1 wherein one of the fastener elements is in the form of a graspable tab having one end configured for permanent mounting to the garment and extending to an opposite, free end.

4. The closure of claim 3 wherein the self-adhesive material of the tab is spaced apart from the free end of the tab to form a graspable end region free of the self-adhesive material.

5. The closure of claim 3 wherein the self-adhesive material of the graspable tab has an edge region nearest the free end and loose from the backing.

6. The closure of claim 1 wherein the self-adhesive material substantially covers the flexible foam layer.

7. The closure of claim 1 wherein the self-adhesive material of at least one of the fastener elements includes a thermoset elastomer.

8. The closure of claim 1 wherein the foam layer comprises closed cell foam.

9. The closure of claim 1 wherein both fastener elements include a corresponding flexible foam layer sandwiched between their backings and layers of self-adhesive material

10. The closure of claim 1 wherein the flexible foam layer has a thickness of between about 0.020 and 0.125 inch.

11. The closure of claim 1 wherein the self-adhesive material comprises a natural rubber dissolved in a solvent.

12. The closure of claim 1 wherein the self-adhesive material comprises a rubber dissolved in a solvent.

13. The closure of claim 1 wherein the sheet-form backing, the flexible foam layer, and the layer of self-adhesive material have a combined Gurley stiffness of between about 25 and 100 milligrams.

14. The closure of claim 1 having peel strength of between about 200 and 400 grams.

15. The closure of claim 1 having shear strength of between about 3,000 and 5,000 grams.

16. The closure of claim 1 wherein the layer of self-adhesive material is releasably engageable with the cooperating fastener element, to provide a multiple cycle fastening.

17. The closure of claim 1 wherein the layers of self-adhesive material have an adhesive strength, when engaged, selected to be greater than a functionally related tear strength of one of the fastener elements, to cause the closure to irreparably tear before separating the self-adhesive material layers.

18. The closure of claim 17 wherein said functionally related tear strength is of the flexible foam layer.

19. The closure of claim 1 further comprising mating hook-and-loop fastener regions adjacent the layers of self-adhesive material and arranged for releasable engagement when the layers of self-adhesive material are engaged.

20. A garment including
a body of flexible sheet material; and
a self-adhesive closure constructed to secure the body in place around a wearer during use, the self-adhesive closure comprising a pair of opposed cooperating fastener elements, each fastener element including
a flexible backing,
a layer of self-adhesive material carried upon the backing and positioned for cooperating engagement with the self-adhesive material of the other fastener element;
at least one of the fastener elements further comprising a flexible foam layer sandwiched between its backing and its layer of self-adhesive material.

21. The garment of claim 20 of a form selected from the group consisting of diapers, incontinence devices, surgical gowns, hats, booties, clean room garments, ankle bands and wrist bands.

22. The garment of claim 20 comprising a diaper.

23. The garment of claim 22 wherein said self-adhesive closure comprises a secondary fastener, with one of the fastener elements disposed above a primary fastener on an outer surface of a front panel of the diaper, and the other of the fastener elements disposed adjacent an upper edge of an inner surface of a rear panel of the diaper.

24. The garment of claim 22 wherein the fastener elements are disposed along lateral edges of the diaper, extending substantially between leg openings and upper diaper edges.

25. The garment of claim 24 wherein the layers of self-adhesive material have an adhesive strength, when engaged, selected to be greater than a functionally related tear strength of one of the fastener elements, to cause the closure to irreparably tear before separating the self-adhesive material layers, and wherein one of the fastener elements extends to a lateral edge of an inner surface of a back panel of the diaper, so as to secure the lateral edge of the inner surface of the back panel of the diaper against grasping when secured to the other of the fastener elements.

26. The garment of claim 20 wherein the layers of self-adhesive material have an adhesive strength, when engaged, selected to be greater than a functionally related tear strength of one of the fastener elements, to cause the closure to irreparably tear before separating the self-adhesive material layers.

27. The garment of claim 26 wherein said functionally related tear strength is of the flexible foam layer.

28. The garment of claim 20 wherein one of the fastener elements is in the form of a graspable tab having one end configured for permanent mounting to the garment and extending to an opposite, free end securable to the other of the fastener elements.

29. A diaper including
a body of flexible sheet material forming front and rear panels with upper edges;
a primary closure for securing the body in place around a wearer during use, the primary closure comprising a graspable tab having one end configured for permanent mounting to the rear panel of the body and having a free end securable to the front panel of the body; and
a secondary closure disposed between the primary closure and the upper edges of the front and rear panels, the secondary closure comprising a pair of opposed cooperating fastener elements, each fastener element having an exposed layer of self-adhesive material positioned for cooperating engagement with the self-adhesive material of the other fastener element.

30. The diaper of claim 29 wherein at least one of the fastener elements of the secondary closure further comprises a flexible foam layer beneath its layer of self-adhesive material.

31. The garment of claim 29 wherein the layers of self-adhesive material have an adhesive strength, when engaged, selected to be greater than a functionally related tear strength of an adjacent portion of the diaper, to cause the diaper to irreparably tear before separating the self-adhesive material layers of the closure.

32. A diaper including
a body of flexible sheet material forming front and rear panels with upper edges; and
an unreleasable adhesive closure constructed to secure the body in place around a wearer during use, the adhesive closure comprising a pair of opposed cooperating fastener elements, each fastener element including an exposed layer of adhesive material positioned for cooperating engagement with the adhesive material of the other fastener element;
the layers of adhesive material having an adhesive strength, when engaged, selected to be greater than a functionally related tear strength of an adjacent portion of the diaper, to cause the diaper to irreparably tear before separating the adhesive material layers of the closure.

33. The diaper of claim 32 wherein at least one of the fastener elements comprises a flexible foam layer beneath its layer of adhesive material.

34. The diaper of claim 33 wherein said functionally related tear strength is of the flexible foam layer.

35. The diaper of claim 32 wherein one of the fastener elements extends to a lateral edge of an inner surface of the rear panel of the diaper.

36. The diaper of claim 32 wherein the adhesive material of each fastener element comprises self-adhesive material.
